(19) United States
(12) Patent Application Publication
(10) Pub. No.: US 2017/0049676 A1
(43) Pub. Date: Feb. 23, 2017

(54) TOPICAL COMPOSITION

(71) Applicant: Conopco, Inc., d/b/a UNILEVER, Englewood Cliffs, NJ (US)

(72) Inventors: Mark John BERRY, Higham Ferrers (GB); Mark lan FOWLER, Huntingdon (GB); Gail JENKINS, Huntingdon (GB); Jennifer Elizabeth POPLE, Rushden (GB); Sally Pamela REDFERN, Sharnbrook (GB)

(73) Assignee: Conopco, Inc., d/b/a UNILEVER, Englewood Cliffs, NJ (US)

(21) Appl. No.: 15/306,891

(22) PCT Filed: Apr. 21, 2015
(86) PCT No.: PCT/EP2015/058574

§ 371 Date: Oct. 26, 2016

(30) Foreign Application Priority Data
May 1, 2014 (EP) 14166785.7

(51) Int. Cl. A61K 8/60; A61K 8/97; A61K 8/66; A61Q 17/04

(52) U.S. Cl. CPC A61K 8/602; A61Q 17/04; A61K 8/97; A61K 8/66

(57) ABSTRACT

The inventors have observed that aqueous mustard seed extract appears to be cytotoxic to fibroblast cells. The cytotoxicity of the aqueous mustard seed extract appears to abate when the extract is heat treated at 120° C. for 15 minutes. The heat treated aqueous mustard seed extract when combined with exogenous myrosinase is not cytotoxic but can induce a modest increase in HO-1 content in cultured fibroblasts and thus would be expected to be useful in a topical composition for treating/preventing itchy skin, particularly the scalp. This invention thus relates to the provision of a topical composition for treating/preventing itchy skin, particularly the scalp.

This invention relates to the provision of a topical composition for treating/preventing itchy skin, particularly the scalp.

Gems et al (Mechanisms of Ageing and Development, 126, 381-387 (2005)) describes the green theory of ageing in which it is suggested that accumulation of lipophilic toxic by-products from stochastic errors of metabolism which are therefore not recognisable to the cell lead to molecular damage and ageing. Gems et al further describe how the smooth endoplasmic reticulum, a eukaryotic organelle, acts as a cellular filter deploying phase 1 and phase 2 metabolism to mobilise and excrete the lipophilic toxins. Phase 1 metabolism results in addition of chemically reactive functional groups which allow further metabolism of otherwise unreactive lipophilic compounds. Phase 2 metabolism involves the addition of side groups which increase solubility aiding excretion. Cytochrome P450 and short-chain dehydrogenases or reductases are agents of phase 1 metabolism in mammalian cells. Uridine 5′-diphospho-glucuronosyltransferases (also known as UDP-glucuronosyltransferases or UGT's), which are glycosyltransferases that catalyze addition of the glycosyl group from a uridine-5′-triphosphate (UTP)-sugar to a small hydrophobic molecule (known as the glucuronidation reaction), are important agents of phase 2 metabolism.

According to Zhang Y (Phase II Enzymes, in Schwab M. (Ed.) Encyclopedia of Cancer: Springer Reference, Springer-Verlag Berlin Heidelberg, 2009), phase 2 enzymes in principle are part of the cellular biotransformation machinery. Cellular biotransformation of xenobiotics and endobiotics may be divided into two sequential phases: phase 1 (oxidation, reduction and hydrolysis reactions) and phase 2 (conjugation reactions). Phase 2 enzymes traditionally refer to the enzymes catalyzing the conjugation reactions, such as glutathione 5-transferase (GST), UDP-glucuronosyltransferase (UGT), N-acetyltransferase (NAT) and sulfotransferase (SULT). However, the term has gradually become broader in scope and is now used to refer to a number of enzymes that catalyze phase 1 reactions, such as heme oxygenase-1 (HO-1). A reason for this ambiguous classification is that many of these enzymes are coordinately induced by a variety of chemical agents through the Kelch-like ECH-associated protein 1 (Keap1)—nuclear factor erythroid 2 related factor 2 (Nrf2)—antioxidant response elements (ARE) signaling pathway, and that all of them play important roles in cytoprotection.

Phase 2 enzymes are major detoxification enzymes and an important part of cellular defense against oxidants and other toxic chemicals.

While each individual phase 2 gene may potentially be subjected to regulation through multiple mechanisms, it is the Keap1-Nrf2-ARE signalling system that is common to many phase 2 genes. Nrf2, nuclear factor erythroid 2-related factor 2, is a transcription factor that is normally sequestered by its repressor Keap1. Binding to Keap1 promotes proteasomal degradation of Nrf2. Dissociation of Nrf2 from Keap1 allows the former to heterodimerize with partners, bind to a cis-acting DNA regulatory element, and promote transcription of the downstream gene. The DNA regulatory element to which the Nrf2 heterodimer binds and activates is termed the antioxidant response element (ARE). One or more copies of the typical ARE sequence is known to exist in the 5′-flanking region of phase 2 genes including HO-1.

Matsushima et al (Inflamm. Res. 58, 705-715 (2009)) reports that mast cells play an important role in allergic inflammatory responses. Quercetin has been shown to inhibit mast cell degranulation and subsequent release of histamine. Heme oxygenase activity was observed to be upregulated after short exposure to quercetin, followed by the induction of HO-1 expression after long exposure to quercetin. The inhibition of degranulation by quercetin was reversed using tin protoporphyrin IX (SnPP), an HO-1 inhibitor. Quercetin was observed to translocate Nrf2 from cytoplasm into nucleus in rat basophilic leukemia (RBL-2H3) cells. These results strongly suggest that quercetin exerted anti-allergic actions via activation of Nrf2-HO-1 pathway.

Kerr et al (Acta Derm Venereol, 91, 404-408 (2011)) reports that in the skin, histamine is synthesised by mast cells that reside in the dermis, as well as by keratinocytes themselves. Histamine is well known as a chemical mediator that plays important roles in allergic inflammatory and immune reactions, and its role in itch has been particularly well established in urticarial reactions, poison ivy and insect bites. However, for skin disorders that result from more complex aetiopathological mechanisms, such as atopic dermatitis, the association between histamine and itch is not definitively established. Nevertheless, increased levels of histamine have been associated with itching in skin disorders ranging from ordinary dry skin to psoriasis. It was observed that the histamine level in subjects with dandruff was more than twice that in those who did not have dandruff. Under conditions known to resolve flaking symptoms, a zinc pyrithione shampoo led to a reduction in histamine in subjects with dandruff to a level that was statistically indistinguishable from those who did not have dandruff. This reduction in histamine was accompanied by a highly significant reduction in the perception of itch intensity. These findings suggested an association between the subjective perception of itch in the scalp and the level of histamine in the skin.

According to Fahey et al (Phytochemistry, 56, 5-51 (2001)), plants of the order Brassicaceae contain organic compounds called glucosinolates. Glucosinolates are water-soluble anions and belong to the glucosides. Every glucosinolate contains a central carbon atom, which is bound via a sulphur atom to the thioglucose group (making a sulfated aldoxime) and via a nitrogen atom to a sulphate group. In addition, the central carbon is bound to a side group; different glucosinolates have different side groups, and it is variation in the side group that is responsible for the variation in the biological activities of these plant compounds. The glucosinolates sinigrin (2-propenyl or allyl glucosinolate) and sinalbin (4-hydroxybenzyl glucosinolate) can be isolated from black (Brassica negra) and white (Sinapis alba) mustard seeds respectively.

In particular, and according to Halkier et al (Annu. Rev. Plant Biol., 57, 303-333 (2006)), there are approximately 120 described glucosinolates, which share a chemical structure consisting of a β-D-glucopyranose residue linked via a sulphur atom to a (Z)-N-hydroximinosulfate ester, plus a variable R group derived from one of eight amino acids. Glucosinolates can be classified by their precursor amino acid and the types of modification to the R group. Compounds derived from Ala, Leu, Ile, Met, or Val are called aliphatic glucosinolates, those derived from Phe or Tyr are called aromatic glucosinolates, and those derived from Trp are called indole glucosinolates. The R groups of most glucosinolates are extensively modified from these precursor amino acids.

Plants accumulating glucosinolates always possess a thioglucoside activity known as myrosinase, which hydrolyzes the glucose moiety on the main skeleton. The products are glucose and an unstable aglycone that can rearrange to form isothiocyanates, nitriles, and other products. Hydrolysis in intact plants appears to be hindered by the spatial separation of glucosinolates and myrosinase or the inactivation of myrosinase, but these components mix together upon tissue damage, leading to the rapid formation of glucosinolate hydrolysis products. Most of the biological activities of glucosinolates are attributed to the actions of their hydrolysis products. Depending on the structure of the side chain and the presence of additional proteins and cofactors, the aglycone then rearranges to form different products, including isothiocyanates, oxazolidine-2-thiones, nitriles, epithionitriles, and thiocyanates.

The most common glucosinolate hydrolysis products in many species are isothiocyanates, which are formed from the aglycone by a Lossen rearrangement involving the migration of the side chain from the oxime carbon to the adjacent nitrogen. When the glucosinolate side chain bears a hydroxyl group at C-2, the isothiocyanates formed are unstable and cyclize to oxazolidine-2-thiones. In a study described by Gil et al (Phytochemistry, 19, 2547-2551 (1980)), the formation of nitriles in vitro is favored at a pH>3 in a model system comprising active thioglucoside glucohydrolase extract prepared from commercial mustard powder and the two glucosinolates allyl glucosinolate and 2-phenethyl glucosinolate.

MacLeod et al (Phytochemistry, 24, 9, 1895-1898 (1985)) describe a study on glucosinolates which possess terminal unsaturation in their side-chain known to hydrolyse to cyanoepithioalkanes. In particular the study involved determining the epithiospecifier protein (ESP) activity in the seeds of two cultivars of Brassica napus, in Brassica campestris and in Lepidium sativum. All four types of seeds contained susceptible substrates for ESP (that is, glucosinolates with terminal unsaturation in their sidechain), although Lepidium sativum contained only a very small amount of one. In the presence of ESP, thioglucoside glucohydrolase enzyme is capable of converting appropriate susceptible glucosinolates to corresponding cyanoepithioalkanes, but in its absence none is formed and only ‘normal’ products such as isothiocyanates and nitriles are obtained. On its own, ESP has no activity. It is thus an enzyme co-factor. It appears that ESP is only absent from systems in which there are definitely no glucosinolates with terminal unsaturation, and the presence of only a very small amount of a susceptible glucosinolate is sufficient for there to be appreciable ESP activity.

Furthermore on addition of relatively small concentrations of ferrous ions, major changes in the relative proportions of hydrolysis products were observed. In all cases, Fe²⁺ caused considerable increases in the relative amounts of 1-cyano-2-hydroxy-3,4-epithiobutane at the expense of 5-vinyloxazolidine-2-thione. Ferrous ions also promote the production of ‘normal’ nitriles (i.e. 1-cyano-2-hydroxybut-3-ene, not the epithionitrile). Remarkably small amounts of added Fe²⁺ were sufficient to bring about a complete reversal of major product distribution. Thus, the addition of a mere 6×10⁻¹¹ mol of Fe²⁺ was enough to change endemic hydrolysis of 2-hydroxybut-3-enylglucosinolate to yield 1-cyano-2-hydroxy-3,4-epithiobutane as the major product (ca. 55%) instead of 5-vinyloxaxolidine-2-thione (ca. 54% in the absence of added Fe²⁺).

Halkier et al (Annu. Rev. Plant Biol., 57, 303-333 (2006) further explains that other hydrolysis products include thiocyanates, which are formed from only three glucosinolates: benzyl-, allyl-, and 4-methylsulfinylbutyl-glucosinolate, all of which form stable side-chain cations. The hydrolysis of indole glucosinolates is somewhat different from that of the other glucosinolate types, because the initially formed isothiocyanates are unstable at neutral or slightly acidic pH, and are converted to further metabolites, including indole-methanols, ascorbic acid conjugates, and oligomeric mixtures.

Bones et al (Phytochemistry, 67, 1053-67 (2006)) reports that thiocyanates are thought to arise from glucosinolates that can give rise to a stable carbocation such as benzyl-, propenyl- and 4-methylthiobutyl-glucosinolates. It has been observed that the sinigrin (2-propenylglucosinolate) aglycone was converted to allyl thiocyanate (3-thiocyanatoprop-1-ene) by T. arvense L. seed meal. Thus it would appear that thiocyanate formation requires at least two enzymes, a myrosinase and a thiocyanate forming factor. It has been reported that seed powder from Lepidium sativum supplemented with ascorbate activates the thiocyanate forming factors.

Shikita et al (Biochem. J. 341, 725-32 (1999)) discloses that an interesting and puzzling feature of most plant myrosinases is that they are strongly activated by ascorbic acid, as well as by several close structural relatives, but not by its oxidation product, dehydroascorbic acid. In the hydrolysis of sinigrin at pH 6.0 by myrosinase from Raphanus sativus var. longipinnatus (daikon), in the absence of ascorbic acid, the purified enzyme hydrolysed sinigrin only very slowly. In the presence of ascorbic acid, enzymic activity was enhanced greatly. However ascorbic acid concentrations greater than 500 μM inhibited the enzyme at 150 μM sinigrin, the reaction velocities with 0.75 and 1.0 mM ascorbic acid were 90 and 73% of those with 500 μM ascorbic acid. Shikita et al also observed that of the products of the myrosinase reaction with sinigrin as substrate, i.e. sulphate, glucose and allyl isothiocyanate, sulphate inhibited activity competitively with respect to both sinigrin and ascorbate. Glucose was only a very weak inhibitor with respect to sinigrin, and allyl isothiocyanate did not significantly inhibit activity at concentrations as high as 1 mM.

Shikita et al also report that most plant myrosinases are activated to some degree by ascorbate. A range of ascorbate concentrations of 0.7-5.0 mM is known for maximal activation of myrosinase in partially purified extracts of six crucifers. The enzyme from Salix alba (white willow) retains significant activity in the absence of ascorbate and is only activated by about 2-3 fold, whereas the enzymes from several other Brassica species show an 8-12 fold activation by ascorbate.

US 2008/0311192 (Kraft Foods Holdings) discloses a particulate composition comprising enteric-coated glucosinolate and beta-thioglucosidase particles. A method of converting glucosinolate to isothiocyanate in the small intestine comprising orally administering to a subject an enteric-coated chemoprotectant precursor composition comprising enteric-coated glucosinolate and beta-thioglucosinodase particles is also disclosed. Uncoated glucosinolate and beta-thioglucosinodase particles may be provided in an enteric-coated capsule. Preferably, the glucosinolate is glucoraphanin and the beta-thioglucosidase is myrosinase. The enteric coating targets the compound for release in the small intestine where beta-thioglucosinodase enzyme converts glucosinolate to chemoprotectant isothiocyanate. In Example 1, myrosinase particles are prepared from lactose, microcrystalline cellulose and white mustard seed extract. Glucosinolate particles are prepared from lactose, microcrystalline cellulose and glucosinolate. The particles were then coated with shellac.

GB 935 629 B (Unstead-Joss et al.) discloses a process for the preparation of a stable mustard rubefacient for external use in which the active principal is preserved against decay, enabling the user to store ready-prepared mustard rubefacient or other mustard preparation for occasional use. The process consists of mixing mustard flour with water or other aqueous (e.g.-oint-ment) type base, and after a specific time interval (during which the natural enzymes of mustard are allowed to act, releasing the active principle), adding a preservative. The preservative action is most effective if compounds of a phenolic nature are added.

GB 2 443 036 B (Engelhard-Lyon) discloses the use of active ingredients preventing, limiting or improving the quality of the derma, notably when the latter is subject to effects of age, notably in a human being. Example 10 discloses preparation of a mustard extract as an active ingredient. Further examples discloses that delivery systems for the active ingredients including a water-in-oil emulsion, a shampoo or shower gel, a lipstick an aqueous gel, a triple emulsion, tablets, an ointment and an injectable formulation.

US 2009/0247477 A1 (John Hopkins School of Medicine) discloses that administration of an isothiocyanate protects against UV light-induced skin carcinogenesis. Sulforaphane analogues and glucosinolates also can be employed. Delivery can be by topical lotion.

GB 612 555 B (Augot) describes the manufacture of mustard plasters comprising a mixture of linseed oil and mustard flour.

Lee et al. (Food Sci. Biotechnol., 18, 5, 1071-1075 (2009)) describes a study on the effects of microencapsulation with modified starch on allyl isothiocyanate, a known, but volatile, antimicrobial, on delaying the release time. Mustard powder was used as a source of allyl isothiocyanate.

US 2006/0127996 (John Hopkins University) discloses a method of extraction of isothiocyanates into the oil from glucosinolate-containing plants, and a method of preparing products, including pharmaceutical compositions, food or drink products, supplements or additives, skin or hair products, and agricultural products, which contain isothiocyanate oil extracted from glucosinolate-containing plants. Isothiocyanates can react with proteins when in the aqueous phase and thus are stabilised on transfer to the oil phase. The combined stability and non-aqueous properties of the extracted isothiocyanate oil expands the potential for different types of applications.

US 2012/0052175 A1 (Ekanayake et al.) discloses a process for producing an essential oil. The essential oil can be white mustard essential oil. The white mustard essential oil can include a moisture sensitive isothiocyanate compound. The moisture sensitive isothiocyanate compound can be 4-hydroxybenzyl isothiocyanate (4-HBITC). The essential oil can be produced from mustard seed, which can comprise a precursor sinalbin and myrosinase enzyme. The mustard seed can be reduced into a powder. Activation of the myrosinase enzyme by using a water solvent and a promoter to form a slurry can be performed, wherein the myrosinase enzyme catalyzes the production of an essential oil comprising an isothiocyanate from the sinalbin precursor.

U.S. Pat. No. 4,062,979 (McCormick & Company) discloses a mustard flour of controlled pungency obtained by mixing separately prepared mustard flours. A first mustard flour is prepared wherein the enzyme is deactivated but the glucoside is retained, whereas in a second mustard flour the enzyme is retained but the glucoside is substantially eliminated. Both mustard flours, alone, are mild in flavour, but when blended and mixed with water, the enzyme in one splits the glucoside in the other producing the pungent flavoring oil. Thus the degree of mildness versus pungency of a mustard product can be predetermined by blending the two flours. The two mustard flours are produced from mustard seed, dehulled or not and fresh or aged, mixed with water to produce a slurry which is wet milled and spray dried to produce mustard flour. When hot water is used to produce the slurry, an enzyme is deactivated, and the flour contains a characteristic glucoside. When cold water is used to produce the slurry, the enzyme splits the glucoside and produces a pungent volatile oil that is driven off in the spray drying step, but the enzyme survives in the mustard flour.

U.S. Pat. No. 4,496,598 (Sakai et al.) discloses a process for producing an improved mustard seed flour, wherein mustard seeds are de-oiled, subjected to heating under conditions to deactivate myrosinase enzyme in the seeds to reduce pungency, followed by grinding to form a flour of reduced pungency, good flavour, enhanced protein content and enhanced preservability. The pungency of mustard is a result of an active enzyme, myrosinase, which is generated by adding water to the mustard. The myrosinase hydrolyzes to form glycoside sinigrin and allyl isothiocyanates.

FR 2 778 095 A1 (Cretoux) discloses a sealed sachet comprising an impermeable face and a permeable face, and containing a paste. The sachet is for therapeutic or aesthetic use on the human or animals. The paste can comprise mustard flour and water.

WO 2012/037193 (Brassica Protection Products LLC) discloses a topical formulation comprising a phase II enzyme inducer precursor and an activating agent. Skin ailments affect millions of people worldwide and cost billions of dollars in treatment costs. UV and solar radiation can also initiate inflammation and suppress the immune response. The effects of such damage include changing the elasticity and content of skin, accelerating the aging of skin (dermatobeliosis), and causing raised, reddish, rough-textured growths (keratoses). In some cases, too much sun exposure causes skin cells to develop into tumorous growths, which can then become skin cancer. One strategy of fighting cancer is to invoke the activity of phase II enzymes through their inducers. Inducers include monofunctional inducers such as diphenol, thiocarbamate, I,2-dithiol-3-thiones, and isothiocyanates. Isothiocyanates are found in various plants, including those from the Brassicae family and comprising broccoli, cauliflower, kale, brussel sprouts, arugula, cabbage, Chinese cabbage, collards, crambe, daikon, kohlrabi, mustard, red radish, turnip, and watercress. Isothiocyanates are generally produced when their precursors, glucosinolates, are hydrolyzed by the enzyme myrosinase (beta-thioglucoside glucohydrolase). In the plants described, glucosinolate and myrosinase are kept separate. This is possibly to prevent premature hydrolysis of glucosinolates into isothiocyanates. Formulations comprising glucosinolates and myrosinase in separated form are desired to treat and prevent skin ailments as well as other cancerous conditions.

WO 2012/116018 (Caudill Seed Company Inc) discloses a spray dried myrosinase/ascorbate mixture is formed from the steps comprising: providing a source of myrosinase, adding ascorbate to the source of myrosinase to produce a mixture, heating the mixture in a solvent to a temperature of about 40 degrees centigrade or higher, and spray drying the myrosinase/ascorbate mixture. Glucosinolates can be catalytically converted to isothiocyanates by the enzyme myrosinase. Both glucosinolates and myrosinase may be found in many crucifers and are generally higher in concentration in the sprouts and seeds than in the rest of the plant. A well known isothiocyanate is sulforaphane, which is a potent inducer of mammalian detoxification and chemoprotection by inducing Phase 2 enzymatic activity, which are known to protect cells against toxic and neoplastic effects of carcinogens. Glucoraphanin, a glucosinolate, is the precursor to sulforaphane. The yield of sulforaphane from glucoraphanin is reduced by epithiospecifier protein (ESP), which is also present in crucifers with myrosinase. ESP catalyzes the formation of sulforaphane nitrile; this alternative reaction pathway competes with the reaction pathway that creates sulforaphane. One way to deactivate ESP is through heat. Example 1 describes milling broccoli seed containing myrosinase, adding water at 35 degrees centigrade and heating to 74-79 degrees centigrade and holding for 5 minutes. Calcium ascorbate is then added and the mixture incubated for 24 hours at 37 degrees centigrade. The mixture is then spray dried. In Example 2, broccoli seeds containing glucoraphanin are rushed at 132 degrees centigrade and de-fatted in super critical carbon dioxide. The myrosinase/ascorbate powder of Example 1 is then added and mixed at 37.5 degrees centigrade and then spray dried to produce sulforaphane.

WO 2012/074412 (Comvita New Zealand Limited) discloses a cancer chemoprotective product, in particular a product containing both a source of glucoraphanin and/or glucoraphanen compound and myrosinase enzyme and which is stable yet provides chemoprotective activity to a subject on administration. Depending on the structure of the specific glucosinolates and the existing reaction conditions, isothiocyanates and nitriles usually constitute the majority of these aglucons. One of the reaction products from the enzymatic reaction of glucoraphanin is sulforaphane. Sulforaphane nitrile has recently been shown not to possess the anticarcinogenic properties of sulforaphane. The isothiocyanate from glucoraphanen is termed sulforaphene. Recent evidence suggests that nitrile formation from glucosinolates is controlled by a heat-sensitive protein, epithiospecifier protein (ESP). Example 3 reports that incubation trials with broccoli seed as the myrosinase source showed a high dependence on the incubation temperature due to the presence of ES protein. Higher incubation temperatures exhibited an increase in the amounts of sulforaphane however lower temperatures appear to steer the reaction towards nitrile formation that are inactive.

US 2010/0317518 (Stevens et al.) discloses that glucosinolate-derived compounds have been used as fungicides, insecticides, bacteriostatic or bactericidal agents, cosmetic additives, and cosmeceutical and/or pharmaceutical agents (e.g., cancer, chemoprotectant, anti-aging, bacteriostatic, bactericidal, treatment and/or prevention of ulcers, treatment and/or prevention of gastritis, treatment of skin disorders including but not limited to eczema, facial eczema, dermatitis, external ulcers, welts, rashes, insect bites, allergic reactions and other irritations, burns, wounds, psoriasis, acneiform eruptions, dryness, dry skin, irritation, skin atrophy, secondary infections and the like). Also disclosed are methods for converting glucosinolate in a glucosinolate-containing plant material to glucosinolate breakdown products (GBPs), comprising: providing an amount of processed glucosinolate-containing plant material, the processed material being depleted of oil and glucosinolate converting enzyme activity by virtue of said processing; providing an amount of glucosinolate converting enzyme activity; mixing the processed material with the amount enzyme activity; hydrating the mixture; and incubating the hydrated mixture, wherein the glucosinolates are enzymatically converted to GBPs. Preferably, the processed plant material comprises a oilseed-derived seedmeal material from which the oil has been removed by the processing (e.g., solvent extraction and/or heat treatment). In particular embodiments, the glucosinolate converting enzyme activity comprises at least one of a myrosinase activity and a nitrile-forming activity. Additional aspects provide low-fat compositions (e.g., herbicide, fungicide, insecticide, bacteriostatic or bactericidal, cosmetic, cosmeceutical or pharmaceutical) comprising GBPs derived from a glucosinolate-containing plant material.

DE 10 308 298 (Kullmer) discloses a herbal pharmaceutical preparation from Brassica ingredients for preventing/treating cancer. A process for obtaining the preparation is described comprising the steps of (a) providing at least one material from a glucosinolate containing plant species, (b) inactivating the endogenous thioglucosidase in the material, (c) extracting the glucosinolates contained in the material, and (d) enzymatically hydrolyzing the glucosinolates to isothiocyanates. Step (b) can be achieved by heating the material to 95-100 degrees centigrade for a sufficient time, typically 1-420 seconds). Step (d) is preferably carried out by at least one exogenous thioglucosidase/glucohydrolase, preferably myrosinase from Raphanus stivus or Sinapis alba.

US 2003/0091518 (Cognis Corporation) discloses a cosmetic and/or pharmaceutical preparation in which the ingredients, glucoraphanin and sulphoraphane, do not produce any skin irritation among users and would activate special repair and detoxification enzymes (for example glutathione-S-transferase), stimulate or regulate cell growth, influence the metabolic activity of fibroblasts or keratinocytes. In Example H3, 5.2 g of a freeze-dried hot aqueous methanol extract of 3.8 day old broccoli buds (involving grinding frozen broccoli buds, dispersing them in pure water, before addition of methanol, and refluxing for 1 hour) were suspended in 100 g of water and the resulting suspension was adjusted to pH 6 by addition of 5N sodium hydroxide solution. 25 U thioglucosidase (1 U=quantity of enzyme needed to prepare 1 μmol of glucose from sinigrin at 25 degrees centigrade (pH 6)) and 9.9 mg of sodium ascorbate were added and the mixture was stirred for 8 hours at 37 degrees centigrade. The pH was then readjusted to pH 6 by addition of more sodium hydroxide and the suspension was filtered and freeze-dried. The isothiocyanate content of the extract was 230 μmol/g.

### SUMMARY OF THE INVENTION

The inventors have observed that aqueous mustard seed extract appears to be cytotoxic to fibroblast cells. The cytotoxicity of the aqueous mustard seed extract appears to abate when the extract is heat treated at 120° C. for 15 minutes. The heat treated aqueous mustard seed extract when combined with exogenous myrosinase is not cytotoxic but can induce a modest increase in HO-1 content in cultured fibroblasts and thus would be expected to be useful in a topical composition for treating/preventing itchy skin, particularly the scalp.

Thus in a first aspect of the invention a topical composition is provided, the topical composition comprising:
(a) ground glucosinolate-containing plant material;
(b) an enzyme source comprising thioglucosidase;
(c) water; and
(d) a compound selected from the group consisting of humectants, organic solvents including petroleum jelly, silicones, perfume, organic/inorganic sunscreens;
   wherein the glucosinolate-containing plant material is heat treated at a temperature of at least 100 degrees centigrade at atmospheric pressure for at least 5 minutes before grinding; and wherein the combination of (a), (b) and (c) only combine on use of the topical composition and wherein the glucosinolate-containing plant material is selected from the group of families consisting of Bataceae, Brassicaceae, Bretschneideraceae, Capparaceae, Caricaceae, Euphorbiaceae, Gyrostemonaceae, Limnanthaceae, Moringaceae, Pentadiplandraceae, Phytolaccaceae, Pittosporaceae, Resedaceae, Salvadoraceae, Tovariaceae, Tropaeolaceae, Akaniaceae, Cleomaceae, Emblingiaceae, Koeberliniaceae, Setchellanthaceae and mixtures thereof, preferably Brassicaceae, most preferably the species Brassica juncea.

### DETAILED DESCRIPTION OF THE INVENTION

In a first aspect of the invention, a topical composition is provided, the topical composition comprising:
(a) ground glucosinolate-containing plant material;
(b) an enzyme source comprising thioglucosidase;
(c) water; and
(d) a compound selected from the group consisting of humectants, organic solvents including petroleum jelly, silicones, perfume, organic/inorganic sunscreens;
   wherein the glucosinolate-containing plant material is heat treated at a temperature of at least 100 degrees centigrade for at least 5 minutes before grinding; and
   wherein the combination of (a), (b) and (c) only combine on use of the topical composition;
   and wherein the glucosinolate-containing plant material is selected from the group of families consisting of Bataceae, Brassicaceae, Bretschneideraceae, Capparaceae, Caricaceae, Euphorbiaceae, Gyrostemonaceae, Limnanthaceae, Moringaceae, Pentadiplandraceae, Phytolaccaceae, Pittosporaceae, Resedaceae, Salvadoraceae, Tovariaceae, Tropaeolaceae, Akaniaceae, Cleomaceae, Emblingiaceae, Koeberliniaceae, Setchellanthaceae and mixtures thereof, preferably Brassicaceae, most preferably the species Brassica juncea. More preferably the glucosinolate-containing plant material comprises a glucosinolate selected from the group consisting of sinigrin, glucoraphanin, benzyl glucosinolate, phenethyl glucosinolate, alpha-naphthyl glucosinolate and mixtures thereof.

Preferably the glucosinolate-containing plant material is heat treated at ambient pressure (i.e. the atmospheric pressure conditions prevailing at the time of heating). In SI units, atmosphere pressure is equal to 101325 Pa, however in the context of the present application, ambient pressure simply means that heat treatment is not carried out under specifically or artificially elevated or reduced pressure.

It is preferred that the glucosinolate-containing plant material does not comprise exclusively glucosinolates bearing a hydroxyl group on the side group, more preferably a hydroxyl group at C2 on the side group, because it has been observed that such glucosinolates form unstable isothiocyanates which cyclise to oxazolidine-2-thiones.

It is essential that the enzyme source does not adventitiously comprise any cytotoxic material. By cytotoxic is meant that at a thioglucosidase level of 0.001 U/6 ml, cytotoxity does not exceed 180, preferably 150, most preferably 120% of the vehicle control when using the cytotoxicity assay set forth herein (using the ApoTox-Glo™ Triplex Assay (Promega Corporation)). Furthermore in one embodiment, the enzyme source comprises thioglucosidase as the sole enzyme, because in the presence of epithiospecifier protein, it has been observed that thioglucosidase is capable of converting glucosinolates with terminal unsaturation to their corresponding cyanoepithioalkanes, rather than the isothiocyanates.

Humectants of the polyhydric alcohol type may be employed in the topical compositions of the invention. The humectant often aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.2 to 25, and preferably from about 0.5 to about 15% w/w of the topical composition including all ranges subsumed therein.

Illustrative and non-limiting examples of the types of organic solvents suitable for use in the present invention include alkanols like ethyl and isopropyl alcohol, mixtures thereof or the like.

Perfumes may be used in the topical composition of the invention. Illustrative non-limiting examples of the types of perfume that may be used include those comprising terpenes and terpene derivatives like those described in Bauer, K., et al., Common Fragrance and Flavor Materials, VCH Publishers (1990). Illustrative yet non-limiting examples of the types of perfume that may be used in this invention include myrcene, dihydromyrenol, citral, tagetone, cis-geranic acid, citronellic acid, mixtures thereof or the like. Preferably the amount of perfume employed in the topical composition of this invention is in the range from 0.000001 to 10, more preferably 0.00001 to 5, most preferably 0.0001 to 2% w/w of the topical composition and including all ranges subsumed therein.

Sunscreens include those materials commonly employed to block ultra-violet radiation. Illustrative organic sunscreens are the derivatives of para-aminobenzoic acid (PABA), cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trade marks, Parsol MCX™ and Benzophenone-3™, respectively. The exact amount of sunscreen employed in the topical compositions of the invention can vary depending upon the degree of protection desired from the sun's ultra-violet radiation. Inorganic sunscreens that reflect or scatter the sun's rays may also be employed. These include oxides like zinc oxide and titanium dioxide.

The pH of the topical composition is typically at least 3, preferably 3 to 8, most preferably 4 to 7.5, because it has been observed in vitro that the formation of nitriles was favoured in a system comprising thioglucosidase extract prepared from mustard powder in the presence of allyl and 2-phenethyl glucosinolates.

The topical composition preferably comprises substantially no ferrous ions, because in their presence, it has been observed that there are significant changes in the proportions of hydrolysis products.

The topical composition preferably comprises 0.1 to 10, preferably 0.3 to 5, most preferably 0.3 to 3 mM vitamin C, because vitamin C has been observed to activate thioglucosidase.

The topical composition preferably comprises glucosinolate-containing plant material in an amount to provide a final concentration in the topical composition of 0.05 to 1000, preferably 0.1 to 500, most preferably 0.5 to 100 mM glucosinolate.

The topical composition preferably comprises an enzyme source comprising thioglucosidase in an amount to provide a final concentration in the topical composition of at least 0.001 U, more preferably at least 0.002 U, more preferably still at least 0.01 U, most preferably at least 0.1 U and preferably at most 20 U, more preferably at most 10 U, more preferably still at most 2 U, most preferably at most 1 U. The enzyme unit (U) is a unit for the amount of a particular enzyme. One U is defined as the amount of the enzyme that catalyzes the conversion of 1 micro mole of substrate per minute under specified conditions. 1 U=1/60 micro katal=16.67 nano katal.

In a second aspect of the invention, a method for treating or preventing itchy skin, preferably the scalp, is provided, the method comprising the step of topically applying the topical composition of the first aspect of the invention.

In one embodiment thererof, a method for treating or preventing itchy skin, preferably the scalp, is provided, the method comprising the step of topically applying to a person in need thereof the topical composition of the first aspect of the invention.

In a further embodiment thereof, the topical composition of the first aspect is provided, for use as a medicament, in particular for use treating or preventing itchy skin, preferably the scalp.

In yet another embodiment thereof, use of the topical composition of the first aspect is provided for use as a medicament, in particular for use treating or preventing itchy skin, preferably the scalp.

In another embodiment thereof, the topical composition of the first aspect is provided, for use in the manufacture of a medicament for use in treating or preventing itchy skin, preferably the scalp.

### EXAMPLES

### Example 1

### Up-Regulation of Heme Oxygenase 1 (HO-1) in Primary Human Neonatal Dermal Fibroblast Cells Treated with an Aqueous Mustard Seed (Brassica juncea) Extract and Exogenous Myrosinase

In this example, primary human neonatal dermal fibroblast cells were treated with an optionally heat treated aqueous extract of mustard seed from Brassica juncea (mustard greens, Indian mustard, Chinese mustard, or leaf mustard, oriental mustard) and exogenous myrosinase to determine the effect of this treatment on heme oxygenase 1 levels.

### Materials

Mustard seed from the Forge cultivar of Brassica juncea

Allyl isothiocyanate (AITC) (Sigma-Aldrich Company)

Singrin (Sigma-Aldrich Company)

Myrosinase>100 U/g solid (also known as thioglucoside glucohydrolase, sinigrinase and sinigrase) (Sigma-Aldrich Company; 1 U will produce 1.0 μmoles glucose per minute from sinigrin at 25 degrees centigrade at pH 6.0)

Primary human neonatal dermal fibroblast cells (Cell Research Corporation)

Sulforaphane (Sigma-Aldrich Company)

### Protease Inhibitor Tablets (Roche Diagnostics GmbH, Complete™ Mini, EDTA-Free Tablets)

### Methods

### Preparation of Mustard Seed Extract

The mustard seed was ground using a mortar and pestle and the crushed seed sieved to produce flour of a maximum particle size of 250 μm. An aqueous extract was prepared at a concentration of 100 mg mustard flour per ml water. The extract was then left to stand at room temperature for 10-15 minutes prior to filtration through glass wool and then a 0.45 μm PTFE syringe filter.

Heat treated mustard seed aqueous extracts were prepared by pre-heating water at the required temperature (50, 60, 70, 80 and 120° C.), adding mustard flour, heating the extract for 15 minutes at the same required temperature, cooling the extract to room temperature, and filtering as described above.

All extracts were frozen at −80° C. until use. Allyl isothiocyanate (AITC) (8.4 mM glucosinolate equivalent assuming complete conversion of all glucosinolate to allyl isothiocyanate) and 8.4 mM glucosinolate singrin controls were also prepared in DMEM supplemented with 10% FBS, known as complete medium.

### Preparation of Myrosinase Solution

A stock solution of exogenous myrosinase was prepared at a concentration of 10 U/ml in water. The enzyme was left to stand in solution at room temperature for 4 hours, and then stored overnight in a fridge before being applied directly to cells.

### Culture of Fibroblast Cells

Primary human neonatal dermal fibroblast cells were cultured and passaged in Dulbecco's Modified Eagle Medium (DMEM) supplemented with 10% foetal bovine serum (FBS), known as complete medium. Cells were routinely plated out in 24-well tissue culture plates at a seeding density of 40,000 cells per well in 1 ml complete medium, and incubated at 37° C. in 5% CO₂ for 48 hours before addition of the test solutions.

### Addition of Test Samples

Test samples were prepared by adding sufficient of the myrosinase solution such that a 6 ml mustard seed extract comprised 1 U myrosinase. This mixture was then diluted 1 in 10, 1 in 25, 1 in 50, and 1 in 100 with DMEM supplemented with 10% FBS. Dermal fibroblasts were treated with 500 ul/well of the test sample for a period of 24 hours. A vehicle and positive controls (2 μM sulforaphane) were included in each experimental plate.

### Harvesting Samples

Any change in cell morphology was noted before the cells were harvested. All tissue culture supernatant was immediately stored at −20° C. The tissue culture supernatant was subsequently assayed for cell viability, cytotoxicity and apoptosis.

### Cytotoxicity Assay

All tissue culture supernatant was examined for cell cytotoxicity using the ApoTox-Glo™ Triplex Assay (Promega Corporation) used according to the manufacturer's instructions. This assay combines three assay chemistries to assess viability, cytotoxicity and apoptosis events in the same cell-based assay well.

First, viability and cytotoxicity are determined by measuring two differential protease biomarkers simultaneously with the addition of a single nonlytic reagent containing two peptide substrates. The live-cell protease activity is restricted to intact viable cells and is measured using a fluorogenic, cell-permeant peptide substrate (GF-AFC Substrate). The substrate enters intact cells, where it is cleaved to generate a fluorescent signal proportional to the number of living cells. This live-cell protease activity marker becomes inactive upon loss of membrane integrity and leakage into the surrounding culture medium. A second, cell-impermeant, fluorogenic peptide substrate (bis-AAF-R110 Substrate) is used simultaneously to measure dead-cell protease activity that has been released from cells that have lost membrane integrity. This results in ratiometric, inversely correlated measures of cell viability and cytotoxicity. The ratio of viable cells to dead cells is independent of cell number and, therefore, can be used to normalize data.

A second reagent containing luminogenic DEVD-peptide substrate for caspase-3/7 (the sequence DEVD (amino acid sequence Asp-Glu-Val-Asp) corresponds to a sequence within PARP1 (poly(ADP-ribose) polymerase 1), a DNA repair enzyme which is cleaved by the protein caspase-3 during cell death by apoptosis) and Ultra-Glo™ Recombinant Thermostable Luciferase is added. Caspase-3/7 cleavage of the substrate releases luciferin, which is a substrate for luciferase and generates light. The light output, measured with a luminometer, correlates with caspase-3/7 activation as a key indicator of apoptosis.

96-well assay plates containing <20,000 cells per well in medium were set up. The tissue culture supernatant and vehicle controls were added to the appropriate wells for a final volume of 100 μl per well. The cells were cultured for 24 hours. Then 20 μl of Viability/Cytotoxicity Reagent containing both the fluorogenic, cell-permeant, peptide substrate glycylphenylalanyl-aminofluorocoumarin (GF-AFC) Substrate and the fluorogenic peptide substrate bis-alanyl-alanyl phenylalanyl-rhodamine 110 (bis-AAF-R110) Substrate was added to each well, and briefly mix by orbital shaking (300-500 rpm for ˜30 seconds). The cells were the incubated for 2.5 hours at 37° C., and the fluorescence measured at 400 nm for excitation and 505 nm for emission (viability), and 485 nm for excitation and 520 nm for emission (cytotoxicity).

Then 100 μl of Caspase-Glo® 3/7 Reagent was added to all the wells, and briefly mix by orbital shaking (300-500 rpm for ˜30 seconds), after which the cells were incubated for 30 minutes at room temperature. Luminescence was measured to determine the level of caspase activation which is a hallmark of apoptosis.

### Preparation of Cell Lysate

After removal and storage of the tissue culture supernatant, the cell monolayer was washed with 1 ml of Dulbecco's Phosphate Buffered Saline (DPBS) per well and lysed with 200 μl cell lysis buffer per well. The cell lysis buffer consisted of 0.5% w/w Triton™ X100 (octyl phenol ethoxylate) and 1 mM ethylenediaminetetraacetic acid (EDTA) in phosphate buffer solution (PBS) at pH 7.2. Protease inhibitor tablets were added to the lysis buffer immediately prior to use, at the manufacturer's recommended level. The plates received one freeze thaw cycle to ensure complete cell lysis. The lysates were subsequently clarified by scraping the samples off the plates with a pipette tip and passing them through an Acrowell™ filter plate (Pall Corporation) using an Acroprep™ vacuum manifold (Pall Corporation) into a 96 well microwell plate. The clarified lysates were stored at −20° C. until needed.

### Total Protein Assay

The total protein concentration of each cell lysate was measured using the Pierce™ BCA protein assay kit (Perbio Science UK Ltd). The BCA protein assay combines the well-known reduction of Cu²⁺ to Cu¹⁺ by protein in an alkaline medium with the highly sensitive and selective colorimetric detection of the cuprous cation (Cu¹⁺) by bicinchoninic acid (BCA). The first step is the chelation of copper with protein in an alkaline environment to form a light blue complex. In this reaction, known as the biuret reaction, peptides containing three or more amino acid residues form a coloured chelate complex with cupric ions in an alkaline environment containing sodium potassium tartrate.

In the second step of the colour development reaction, bicinchoninic acid reacts with the reduced (cuprous) cation that was formed in step one. The intense purple-coloured reaction product results from the chelation of two molecules of BCA with one cuprous ion. The BCA/copper complex is water-soluble and exhibits a strong linear absorbance at 562 nm with increasing protein concentrations. The BCA reagent is approximately 100 times more sensitive (lower limit of detection) than the pale blue colour of the first reaction.

The reaction that leads to BCA colour formation is strongly influenced by four amino acid residues (cysteine or cystine, tyrosine, and tryptophan) in the amino acid sequence of the protein. As the universal peptide backbone also contributes to colour formation, this helps to minimize variability caused by protein compositional differences.

A set of eight standard solutions ranging from 0 to 800 μg/ml protein was prepared from the supplied 2 mg/ml bovine serum albumin (BSA) stock solution. 10 μl of standard or cell lysate was added to duplicate wells of a flat-bottomed, 96-well microtitre plate. The reagent solution was prepared according to the kit instructions from 50 parts reagent A and 1 part reagent B. 200 μl of the final reagent was added to each well of the microtitre plate. The plate was mixed, covered and incubated at 37° C. for 30 minutes and absorbance read at 540 nm. A protein standard curve was constructed and used to determine the protein concentration of each individual cell lysate. The data for total protein was used to normalise the data from the heme oxygenase 1 assay below.

### Heme Oxygenase 1 (HO-1) Assay

The heme oxygenase 1 (HO-1) protein concentration of each cell lysate was assayed using the Human Total HO-1/HMOX1 DuoSet IC assay (R&D Systems Europe Ltd) according to the manufacturer's instructions. This DuoSet IC ELISA (indirect competitive enzyme-linked immunosorbent assay) contains the basic components required for the development of sandwich ELISAs to measure HO-1/HMOX1 (heme oxygenase (decycling) 1 which is a human gene that encodes for the enzyme heme oxygenase 1)/HSP32 (heat shock protein 32 (Hsp32), also known as heme oxygenase 1 (HO-1)) in cell lysates. An immobilized capture antibody specific for HO-1/HMOX1/HSP32 binds both phosphorylated and unphosphorylated HO-1/HMOX1/HSP32. After washing away unbound material, a biotinylated detection antibody is used to detect both phosphorylated and unphosphorylated protein, utilizing a standard Streptavidin—horse radish peroxidase (HRP) format.

Eight HO-1 standards were prepared in reagent diluent (0.5% w/w Triton™ X100 (octyl phenol ethoxylate) and 1 mM EDTA in PBS at pH 7.2) at concentrations ranging from 0.15625 to 10 ng/ml, including a negative control. The HO-1 capture antibody was diluted to a concentration of 8 μg/ml in PBS and was bound to the microtitre plate (Greiner Bio-One Ltd) overnight at room temperature. Unbound antibody was removed by washing three times with wash buffer (0.05% w/w Tween 20 (Polysorbate 20) in PBS) on an automatic plate washer. The plate was blocked with 300 μl per well of 1% w/w bovine serum albumin (BSA) in PBS for 1 hour and washed three times in wash buffer.

100 μl of cell lysate, diluted 1/5 in reagent diluent, or standard was added to duplicate wells. The plate was incubated at room temperature for 2 hours before being washed three times with wash buffer. 100 μl of HO-1 detection antibody, diluted to a concentration of 200 ng/ml, was added to each well and the plate incubated at room temperature for 2 hours. The plate was washed as before. 100 μl of streptavidin HRP diluted 1/200 in 1° A BSA in PBS was added to each well and incubated in the dark for 20 minutes at room temperature. The plate was washed as before and then 100 μl of substrate solution (1:1 mixture of Colour Reagent A and Colour Reagent B) was added to each well and incubated, in the dark at room temperature until colour developed (approximately 20 minutes). 50 μl of stop solution (2 M aqueous H₂SO₄) was applied to each well and the plate read on a microplate reader (Dynex MRX) at 450 nm with wavelength correction set at 540 nm.

A standard curve was plotted of mean Optical Density versus HO-1 concentration and the line of best fit calculated by regression analysis. The unknown concentration of HO-1 protein in all the samples was estimated from this. The results were normalised using the total protein data obtained from the assay previously described, and expressed as ng HO-1 per ug protein or as percentage change in HO-1 compared to the vehicle control value.

### Results

The results of the cytotoxicty and HO-1 assays are presented in tables 1 to 3 below.

Table 1 shows the HO-1 levels (pg HO-1 per μg protein) of the primary human neonatal dermal fibroblast cells treated with an aqueous mustard seed extract with and without heat treatment for 15 minutes, treated with exogenous myrosinase and appropriate controls (vehicle is water). The figures in brackets indicate the degree of dilution in parts by volume with Dulbecco's Modified Eagle Medium (DMEM) supplemented with 10° A foetal bovine serum (FBS). The myrosinase control was prepared by diluting the stock solution to 0.1 IU/ml with Dulbecco's Modified Eagle Medium (DMEM) supplemented with 10° A foetal bovine serum (FBS).

In the absence of heat treatment, the aqueous mustard extract induced levels of HO-1 of about 400 pg HO-1 per μg protein in the fibroblasts. This level of HO-1 induction was considered to be high. When the mustard extract was heat treated up to 70° C., the level of HO-1 that was induced in the fibroblasts dropped significantly. There seemed to be no effect on HO-1 induction if the mustard extracts were not heat treated or treated below 70° C. and combined with exogenous myrosinase. This suggested the HO-1 induction measured was due to something in the mustard extract and not due to an enzymatic end product produced by combining the extract with exogenous myrosinase. As the temperature of the heat treatment of the mustard was increased to 120° C. only very low levels of HO-1 were induced in the fibroblast cells, It was hypothesised that the high HO-1 induction induced by the mustard extract that was not heat treated or heat treated at temperatures lower than 120° C. was due to a component (enzyme or protein) in the extract causing cellular cytotoxicity. The heat treatment clearly inactivated or somehow removed this product. The amount of HO-1 induced in fibroblasts treated with 120° C. heat treated mustard extract was 75 pg/μg protein which was similar to the vehicle control level at 50 pg/μg protein. This level is the background level of HO-1 protein present in normal fibroblasts in in vitro culture.

When the 120° C. heat treated mustard extract was combined with exogenase myrosinase and applied to the fibroblasts, a higher level of HO-1 induction occurred than for the sample without myrosinase. We hypothesise that this is due to the favourable production of allyl isothiocyanate from the glucosinolate sinigrin in the mustard extract. Allyl isothiocyanate is a known activator of nrf-2 and will induce production of phase II detox enzymes including HO-1. The levels of HO-1 induced in this sample were modest at 176 pg/μg protein, hence we think this induction was due to the production of favourable myrosinase-linked end products and not due to the presence of cytotoxic materials. When only exogenous myrosinase was applied to the fibroblasts, no HO-1 was induced above vehicle levels illustrating the myrosinase alone has no effect.

TABLE 1
HO-1 levels (pg HO-1 per μg protein) of the
primary human neonatal dermal fibroblast
cells treated with an aqueous mustard seed
extract with and without heat treatment for 15
minutes, treated with exogenous myrosinase
and appropriate controls (vehicle is water).
Figures in brackets indicate the degree of dilution
in parts by weight with Dulbecco's Modified
Eagle Medium (DMEM) supplemented with
10% foetal bovine serum (FBS). Myrosinase
control was prepared by diluting the stock solution
to 0.1 IU/ml with Dulbecco's Modified Eagle
Medium (DMEM) supplemented with 10%
foetal bovine serum (FBS). Errors at 95%
confidence limits with n = 3.

| | Mustard seed | |
|---|---|---|
| | extract heat | HO-1 (pg |
| | treatment | HO-1 per |
| | (° C.) | μg protein) |
| Vehicle (1/50) | — | 17.25 ± 4.47 |
| Mustard extract (1/50) | none | 397.87 ± 53.31 |
| Mustard extract + | none | 396.64 ± 81.02 |
| myrosinase solution (1/50) | | |
| Vehicle (1/100) | — | 29.95 ± 8.26 |
| Mustard extract (1/100) | none | 307.02 ± 33.50 |
| Mustard extract + | none | 371.67 ± 21.09 |
| myrosinase solution (1/100) | | |
| Vehicle (1/50) | — | 18.38 ± 10.32 |
| Mustard extract (1/50) | 50 | 421.11 ± 28.44 |
| Mustard extract + | 50 | 443.88 ± 15.31 |
| myrosinase solution (1/50) | | |
| Vehicle (1/100) | — | 46.60 ± 37.87 |
| Mustard extract (1/100) | 50 | 271.42 ± 28.29 |
| Mustard extract + | 50 | 296.96 ± 17.94 |
| myrosinase solution (1/100) | | |
| Vehicle (1/50) | — | 11.67 ± 5.90 |
| Mustard extract (1/50) | 60 | 359.07 ± 7.42 |
| Mustard extract + | 60 | 308.77 ± 19.71 |
| myrosinase solution (1/50) | | |
| Vehicle (1/100) | — | 11.76 ± 1.07 |
| Mustard extract (1/100) | 60 | 192.07 ± 16.85 |
| Mustard extract + | 60 | 193.07 ± 16.82 |
| myrosinase solution (1/100) | | |
| Vehicle (1/50) | — | 4.03 ± 1.16 |
| Mustard extract (1/50) | 70 | 122.76 ± 5.15 |
| Mustard extract + | 70 | 132.78 ± 8.64 |
| myrosinase solution (1/50) | | |
| Vehicle (1/100) | — | 4.03 ± 0.22 |
| Mustard extract (1/100) | 70 | 66.25 ± 10.32 |
| Mustard extract + | 70 | 75.28 ± 3.89 |
| myrosinase solution (1/100) | | |
| Vehicle (1/50) | — | 5.38 ± 1.00 |
| Mustard extract (1/50) | 80 | 120.11 ± 3.16 |
| Mustard extract + | 80 | 102.07 ± 3.03 |
| myrosinase solution (1/50) | | |
| Vehicle (1/100) | — | 7.99 ± 0.40 |
| Mustard extract (1/100) | 80 | 80.73 ± 6.87 |
| Mustard extract + | 80 | 51.25 ± 10.14 |
| myrosinase solution (1/100) | | |
| Vehicle (1/50) | — | 51.78 ± 30.98 |
| Mustard extract (1/50) | 120 | 75.06 ± 47.02 |
| Mustard extract + | 120 | 176.38 ± 14.87 |
| myrosinase solution (1/50) | | |
| Vehicle (1/100) | — | 45.84 ± 20.63 |
| Mustard extract (1/100) | 120 | 57.90 ± 24.37 |
| Mustard extract + | 120 | 275.19 ± 11.70 |
| myrosinase solution (1/100) | | |
| Myrosinase solution (1/50) | — | 6.87 ± 3.15 |
| Myrosinase solution (1/100) | — | 8.41 ± 2.50 |

Table 2 shows the cytotoxicity (Relative Fluorescence Units (RFU)×1000 of ratio of levels of excitation at 485 nm to emission at 520 nm) of aqueous mustard seed extract on the primary human neonatal dermal fibroblast cells with and without heat treatment, and appropriate controls (vehicle is water). The figures in brackets indicate the degree of dilution in parts by weight with Dulbecco's Modified Eagle Medium (DMEM) supplemented with 10% foetal bovine serum (FBS). The allyl isocyanate and sinigrin controls were prepared in Dulbecco's Modified Eagle Medium (DMEM) supplemented with 10% foetal bovine serum (FBS).

The cytotoxicity of the aqueous mustard seed extract was observed to be significant dependant on its concentration and varying from approximately 2000 to 300 RFU (×1000) for 1/10 to 1/100 dilutions. The degree of cell cytotoxocity of the aqueous mustard seed extract at the same concentrations was observed to drop dramatically on heat treatment at 120° C. for 15 minutes to approximately 112 to 77 RFU (×1000) for 1/10 to 1/100 dilutions. We hypothesis that this shows the mustard extract that is not heat treated contains a component that is cytotoxic and this was driving a very high level of HO-1 induction in the fibroblasts as a result. When the mustard extract was heat treated at 120° C., this cytoxicity was removed and the extract showed very little or no induction of HO-1. It is hypothesised that the heat treatment may have inactivated an enzyme or denatured a protein of unknown origin. Neither the glucosinolate, isothiocyanate or myrosinase enzyme on their own induced any cytotoxicity suggesting the cytotoxic component of the mustard extract was not any of these components.

TABLE 2
Cytotoxicity (Relative Fluorescence Units (RFU) × 1000
of ratio of levels of excitation at 485 nm to emission
at 520 nm) of aqueous mustard seed extract on the
primary human neonatal dermal fibroblast cells with and
without heat treatment, and appropriate controls
(vehicle is water). Figures in brackets indicate the
degree of dilution in parts by weight Dulbecco's
Modified Eagle Medium (DMEM) supplemented with
10% foetal bovine serum (FBS). Allyl isocyanate
and sinigrin controls were prepared in Dulbecco's
Modified Eagle Medium (DMEM) supplemented
with 10% foetal bovine serum (FBS). Myrosinase
control was prepared by diluting the stock
solution to 0.1 IU/ml with Dulbecco's Modified
Eagle Medium (DMEM) supplemented with
10% foetal bovine serum (FBS). Errors at
95% confidence limits with n = 4.

| | Mustard seed | |
|---|---|---|
| | extract heat | |
| | treatment | Cytotoxicity |
| | (° C.) | (RFU × 1000) |
| No cells | — | 2.49 ± 0.05 |
| Untreated cells | — | 63.96 ± 4.66 |
| Vehicle (1/10) | — | 141.87 ± 4.79 |
| Vehicle (1/25) | — | 92.55 ± 4.98 |
| Vehicle (1/50) | — | 81.35 ± 9.92 |
| Vehicle (1/100) | — | 79.64 ± 8.95 |
| Mustard extract (1/10) | none | 1970.65 ± 4.84 |
| Mustard extract (1/25) | none | 1058.59 ± 169.67 |
| Mustard extract (1/50) | none | 508.83 ± 124.17 |
| Mustard extract (1/100) | none | 277.09 ± 41.43 |
| Mustard extract (1/10) | 120 | 112.49 ± 8.69 |
| Mustard extract (1/25) | 120 | 86.02 ± 6.64 |
| Mustard extract (1/50) | 120 | 82.60 ± 9.08 |
| Mustard extract (1/100) | 120 | 77.36 ± 7.75 |
| Untreated cells | | 18.63 ± 1.41 |
| Allyl isocyanate control (1/50) | — | 66.71 ± 4.63 |
| Sinigrin control (1/50) | — | 80.94 ± 39.75 |
| Myrosinase solution (1/5) | — | 38.88 ± 2.58 |
| Myrosinase solution (1/10) | — | 41.82 ± 1.22 |
| Vehicle (1/10) | — | 38.85 ± 1.93 |

Table 3 shows normalised HO-1 levels (pg HO-1 per μg protein wherein vehicle=100) of the primary human neonatal dermal fibroblast cells treated with an aqueous mustard seed extract with heat treatment, treated with exogenous myrosinase and appropriate controls (vehicle is water). The figures in brackets indicate the degree of dilution in parts by weight with Dulbecco's Modified Eagle Medium (DMEM) supplemented with 10% foetal bovine serum (FBS). The myrosinase control was prepared by diluting the stock solution to 0.1 IU/ml with Dulbecco's Modified Eagle Medium (DMEM) supplemented with 10% foetal bovine serum (FBS). The sinigrin control was prepared in Dulbecco's Modified Eagle Medium (DMEM) supplemented with 10% foetal bovine serum (FBS).

Addition of exogenous myrosinase to heat treated aqueous mustard seed extract led to up-regulation of HO-1 from 1.5× to 3.3×the vehicle control. The response was similar to that seen with allyl isocyanate.

TABLE 3
Normalised HO-1 levels (μg HO-1 per
μg protein wherein vehicle = 100) of the primary
human neonatal dermal fibroblast cells treated with
an aqueous mustard seed extract with heat treatment,
treated with exogenous myrosinase and appropriate
controls (vehicle is water). Figures in brackets indicate
the degree of dilution in parts by weight with Dulbecco's
Modified Eagle Medium (DMEM) supplemented with
10% foetal bovine serum (FBS). Myrosinase control was
prepared by diluting the stock solution to 0.1 IU/ml with
Dulbecco's Modified Eagle Medium (DMEM) supplemented
with 10% foetal bovine serum (FBS). Sinigrin control
was prepared in Dulbecco's Modified Eagle Medium
(DMEM) supplemented with 10% foetal bovine serum
(FBS). Errors at 95% confidence limits with n = 3

| | Mustard seed | Normalised |
|---|---|---|
| | extract heat | HO-1 |
| | treatment | (vehicle = |
| | (° C.) | 100) |
| Vehicle (1/50) | — | 100 ± 59.82 |
| Mustard extract (1/50) | 120 | 144.96 ± 90.81 |
| Mustard extract + | 120 | 340.63 ± 28.72 |
| myrosinase solution (1/50) | | |
| Allyl isocyanate control (1/50) | — | 335.46 ± 54.80 |
| Sinigrin control (1/50) | — | 168.32 ± 73.93 |
| Myrosinase solution | — | 58.86 ± 27.00 |
| Vehicle (1/100) | — | 100 ± 31.52 |
| Mustard extract (1/100) | 120 | 126.32 ± 53.18 |
| Mustard extract + | 120 | 600.39 ± 25.52 |
| myrosinase solution (1/100) | | |
| Allyl isocyanate control (1/100) | — | 383.74 ± 30.02 |
| Sinigrin control (1/100) | — | 49.08 ± 12.98 |
| Myrosinase solution | — | 71.48 ± 21.24 |

### Conclusions

Tables 1 and 2 show that aqueous mustard seed extract appears to be cytotoxic to the fibroblast cells and because of this cytotoxicity induces a very high level of HO-1. Neither myrosinase, nor the glucosinolate sinigrin, nor allyl isothiocyanate induce cytotoxicity on their own (at comparable levels), so the cytotoxicity is not due to the presence of these materials in the mustard extract. The cytotoxicity of the aqueous mustard seed extract appears to abate when the extract is heat treated at 120° C. for 15 minutes suggesting possibly an unknown enzyme is inactivated or protein denatured which removes this inherent cytoxicity. The heat treated aqueous mustard seed extract when combined with exogenous myrosinase is not cytotoxic but can induce a modest increase in HO-1 content in cultured fibroblasts. This may suggest that glucosinolates within the heat treated extract are being converted to favourable end products, such as allyl isothiocyanate and these end products are causing a modest increase in the HO-1 content of the cells.

### Example 2

### Up-Regulation of Heme Oxygenase 1 (HO-1) in Primary Human Neonatal Dermal Fibroblast Cells Treated with Selected Exogenous Isothiocyanate Compounds

In this example, primary human neonatal dermal fibroblast cells were treated with selected exogenous isothiocyanate compounds to determine their effects on heme oxygenase 1 levels.

### Materials (Additional)

Alpha-naphthyl isothiocyanate (NITC) (Sigma-Aldrich Company)

Phenethyl isothiocyanate (PEITC) (Sigma-Aldrich Company)

Sulforaphane (Sigma-Aldrich Company)

Benzyl isothiocyanate (BITC) (Sigma-Aldrich Company)

Primary human adult dermal fibroblast cells (Cell Research Corporation)

### Methods

### Culture of Fibroblast Cells

Primary human adult dermal fibroblast cells were routinely cultured and passaged in Dulbecco's Modified Eagle Medium (DMEM) supplemented with 10% foetal bovine serum (FBS), known as complete medium. Cells were plated out in 12-well tissue culture plates at a seeding density of 20,000 cells per well in 1 ml of low serum medium (DMEM supplemented with 1% FBS) and incubated at 37° C. in 5% CO2 for 96-120 hours before addition of the test samples.

### Addition of Test Samples

The isothiocynates (all from Sigma-Aldrich Company) sulforaphane, allyl isothiocyanate (AITC), alpha-naphthyl isothiocyanate (NITC), phenethyl isothiocyanate (PEITC) and benzyl isothiocyanate (BITC) were prepared in dimethyl sulphoxide (DMSO) and diluted in DMEM supplemented with 1% FBS to a final concentration of 1 uM and 2 uM. Dermal fibroblasts were treated with 1 ml/well of the test samples for a period of 24 hours. A solvent vehicle was included in each experimental plate.

### Preparation of Cell Lysate

After 24 hours the cell monolayer was washed with 1 ml of Dulbecco's Phosphate Buffered Saline (DPBS) per well and lysed with 400 μl of RIPA cell lysis buffer per well. The RIPA cell lysis buffer consisted of 150 mM NaCl, 1% v/v Nonidet-P40 (Igepal CA603 from Fluke 56741), 0.1% w/v sodium dodecyl sulphate (SDS) and 0.1% w/v sodium deoxycholate in 50 mM Tris-HCL buffer at pH 7.6. Protease inhibitor tablets were added to the lysis buffer immediately prior to use, at the manufacturer's recommended level. The plates received one freeze thaw cycle to ensure complete cell lysis. The lysates were subsequently clarified by scraping the samples off the plates with a pipette tip, centrifuging at 13,000 rpm for 10 minutes and transferring the supernatant into a 96 well microtitre plate. The clarified lysates were stored at −20° C. until needed.

### Total Protein Assay

The total protein concentration of each cell lysate was measured using the Pierce™ BCA protein assay kit (Perbio Science UK Ltd) and hence conducted in accordance with that set forth in Example 1.

### Heme Oxygenase 1 (HO-1) Assay

The heme oxygenase 1 (HO-1) protein concentration of each cell lysate was assayed using the Human HO-1 ELISA Kit from Assay Designs (ADI-EKS-800, Enzo Life Sciences, UK) according to the manufacturer's instructions. Briefly, biopsy samples were diluted 1 in 10 in sample diluent, and 100 μl transferred to the pre-coated anti-HO-1 immunoassay plate. A 7-point recombinant HO-1 standard curve, ranging from 25 to 0.39 ng/ml, was also prepared in sample diluent. The immunoassay plate was incubated at room temperature for 30 minutes, washed six times with Wash Buffer, and incubated for a further 60 minutes at room temperature with 100 μl/well of anti-HO-1 antibody solution. The plate was washed as above and incubated for 30 minutes at room temperature with 100 μl/well of horseradish peroxidase (HRP)-conjugate solution. Again the plate was washed as above and 100 μl/well of 3,3′,5,5′-tetramethylbenzidine (TMB)-substrate added to each well for 15 minutes at room temperature in the dark. Following the addition of 100 μl/l well of stop solution, the absorbance was read at 450 nm on a microplate reader (Dynex MRX) and the unknown lysate levels of HO-1 were extrapolated from the standard curve. The results were normalised using the total protein data obtained from the assay previously described, and expressed as ng HO-1 per ug protein or as percentage change in HO-1 compared to the vehicle control value.

### Results

The results are presented in Table 4 from which it is apparent that each of sulforaphane, allyl isothiocyanate (AITC), alpha-naphthyl isothiocyanate (NITC), phenethyl isothiocyanate (PEITC) and benzyl isothiocyanate (BITC), either at 1 or 2 μM increase the production of HO-1 in primary human adult dermal fibroblast cells.

TABLE 4
HO-1 (ng HO-1 per ug protein) expressed
from primary human adult dermal fibroblast
cells following treatment with a series
of isocyanates and a vehicle control.

| | | HO-1 |
|---|---|---|
| | | (pg HO-1 |
| | | per μg |
| Treatment | Dosage | protein) |
| Vehicle Control | 0.1% DMSO | 179 ± 28 |
| Alpha-naphthyl isothiocyanate (NITC) | 1 μM | 195 ± 25 |
| Alpha-naphthyl isothiocyanate (NITC) | 2 μM | 191 ± 31 |
| Allyl Isothiocyanate (AITC) | 1 μM | 207 ± 13 |
| Allyl Isothiocyanate (AITC) | 2 μM | 243 ± 26 |
| Sulforaphane | 1 μM | 280 ± 15 |
| Sulforaphane | 2 μM | 309 ± 45 |
| Phenethyl isothiocyanate (PEITC) | 1 μM | 429 ± 49 |
| Phenethyl isothiocyanate (PEITC) | 2 μM | 668 ± 101 |
| Benzyl isothiocyanate (BITC) | 1 μM | 381 ± 50 |
| Benzyl isothiocyanate (BITC) | 2 μM | 730 ± 170 |

### Conclusions

All of the isothiocyanates that were tested in this assay increased the production of HO-1 in primary human adult dermal fibroblast cells.

### Example 3

### Preparation of Particles Comprising Hydrogenated Coconut Fat Enrobed Mustard Flour

In this example, particles comprising fat encapsulated heat inactivated mustard seed flour are prepared. Encapsulation isolates the mustard seed flour from the aqueous carrier of a topical personal care composition.

### Ingredients

5 g heat inactivated mustard flour

25 g hydrogenated coconut fat (mp 36-40° C.)

80 g water chilled with ice

### Method

Mustard flour is mixed in weight ratio of 1:5 in warm hydrogenated coconut fat (maintained at 50° C.). The mixture is then placed into a syringe with a 19 gauge needle. The needle is held under the iced water and injected rapidly under the surface creating small fat particles, enrobing the mustard flour, that harden in the chilled water. These particles can then be carefully removed from the chilled water and placed in a myrosinase-containing product base, such as a shampoo or conditioner, at room temperature. In use, body heat or warm water would melt the mustard flour capsules allowing glucosinolates within the mustard flour to react with the myrosinase in the product base converting them to allyl isothiocyanate. This would result in low level in situ production and delivery of allyl isothiocyanate to the skin/scalp leading to the claimed skin benefits from up-regulation of HO-1.

1. A topical composition comprising:
(a) ground glucosinolate-containing plant material;
(b) an enzyme source comprising thioglucosidase;
(c) water; and
(d) a compound selected from the group consisting of humectants, organic solvents including petroleum jelly, silicones, perfume, organic/inorganic sunscreens;
wherein the glucosinolate-containing plant material is heat treated at a temperature of at least 100 degrees centigrade for at least 5 minutes before grinding; and
wherein the combination of (a), (b) and (c) only combine on use of the topical composition;
and wherein the glucosinolate-containing plant material is selected from the group of families consisting of Bataceae, Brassicaceae, Bretschneideraceae, Capparaceae, Caricaceae, Euphorbiaceae, Gyrostemonaceae, Limnanthaceae, Moringaceae, Pentadiplandraceae, Phytolaccaceae, Pittosporaceae, Resedaceae, Salvadoraceae, Tovariaceae, Tropaeolaceae, Akaniaceae, Cleomaceae, Emblingiaceae, Koeberliniaceae, Setchellanthaceae and mixtures thereof, preferably Brassicaceae, most preferably the species Brassica juncea.

2. A topical composition according to claim 1, wherein the glucosinolate-containing plant material comprises a glucosinolate selected from the group consisting of sinigrin, glucoraphanin, benzyl glucosinolate, phenethyl glucosinolate, alpha-naphthyl glucosinolate and mixtures thereof.

3. A topical composition according to claim 1, wherein the glucosinolate-containing plant material does not comprise exclusively glucosinolates bearing a hydroxyl group on the side group.

4. A topical composition according to claim 3, wherein the glucosinolate-containing plant material does not comprise exclusively glucosinolates bearing a hydroxyl group at C2 on the side group.

5. A topical composition according to claim 1, wherein thioglucosidase is the sole enzyme.

6. A topical composition according to claim 1, wherein the pH is at least 3, preferably 3 to 8, most preferably 4 to 7.5.

7. A topical composition according to claim 1, wherein the topical composition comprises substantially no ferrous ions.

8. A topical composition according to claim 1, wherein the topical composition comprises 0.1 to 10, preferably 0.3 to 5, most preferably 0.3 to 3 mM vitamin C.

9. A topical composition according to claim 1 comprising glucosinolate-containing plant material in an amount to provide a final concentration in the topical composition of 0.05 to 1000, preferably 0.1 to 500, most preferably 0.5 to 100 mM glucosinolate.
